(19) United States
(12) Patent Application Publication
(10) Pub. No.: US 2013/0225530 A1
(43) Pub. Date: Aug. 29, 2013

(54) WOGONIN FOR THE PREVENTION AND THERAPY OF CARDIAC HYPERTROPHY

(71) Applicants: Min Li-Weber, Bad Durkheim (DE); H. Peter Krammer, Heidelberg (DE); Gernot Polier, Dossenheim (DE)

(72) Inventors: Min Li-Weber, Bad Durkheim (DE); H. Peter Krammer, Heidelberg (DE); Gernot Polier, Dossenheim (DE)

(73) Assignee: DKFZ DEUTSCHES KREBSFORSCHUNGSZENTRUM, Heidelberg (DE)

(21) Appl. No.: 13/809,312

(22) PCT Filed: Jul. 6, 2011
(86) PCT No.: PCT/EP2011/061367

§ 371 Date: Mar. 27, 2013

(30) Foreign Application Priority Data
Jul. 12, 2010 (EP) 10169195.4

(51) Int. Cl. C07D 413/10; C07D 405/10; A61K 31/5355; A61K 31/352; A61K 31/453; C07D 311/30; A61K 45/06

(52) U.S. Cl. CPC C07D 413/10; C07D 311/30; C07D 405/10; A61K 45/06; A61K 31/352; A61K 31/453; A61K 31/5355
USPC 514/91; 514/456; 514/320; 514/228.8; 514/412; 514/423; 514/221; 514/307; 514/252.12; 549/403; 546/196; 544/96

(57) ABSTRACT

The present invention relates to a pharmaceutical composition for use as a medicament for the treatment or prophylaxis of cardiac hypertrophy, comprising at least one compound of formula (I), wherein: R¹ is e.g. hydrogen, —CH₃, R² is e.g. hydrogen, —CH₃, R⁴ is hydrogen, —OH, —NH₂; R⁵ is hydrogen, —OH; R⁶ is hydrogen, —OH; R³ is e.g. —OCH₃ or a pharmaceutically acceptable salt, and at least one pharmaceutical excipient.

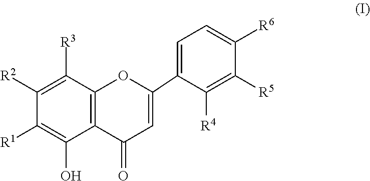

The present invention relates to the small molecule compound Wogonin and its derivatives for the treatment and prevention of cardiac hypertrophy. Cardiac hypertrophy is a serious medical problem for many people in various countries. The treatment comprises reducing one or more symptoms of cardiac hypertrophy, such as reduced exercise capacity, reduced blood ejection volume, increased left ventricular end diastolic pressure, increased pulmonary capillary wedge pressure, reduced cardiac output, cardiac index, increased pulmonary artery pressures, increased left ventricular end systolic and diastolic dimensions, and increased left ventricular wall stress, wall tension and wall thickness, and the same for the right ventricle. In addition, Wogonin and some derivatives can be used to prevent cardiac hypertrophy and its associated symptoms from arising.

In the last years, many efforts have been made to find compounds for the treatment of cardiac diseases. Traditional approaches to suppressing cardiac hypertrophy have focused on outside-in signalling, e.g. to block neurohormones (catecholamines, angiotensin, aldosterone), or calcium triggers (L-type Ca²⁺-channel blockers) or target pathological load (vasodilators and diuretics). However, these treatments vary in effectiveness. Recent research has targeted the signalling inside the cardiac muscle cell that ultimately alter gene and protein expression involved in cell enlargement and chamber remodelling. These pathways have redundancy, as revealed by persistent inducible hypertrophy in animal models in which one or another of the pathways is genetically inhibited.

On a cellular level, cardiac hypertrophy is characterized by a global increase in RNA and protein per cell. Hypertrophic stimuli, such as phenylephrine and endothelin-1 can induce phosphorylation of the RNA polymerase II (RNAPII). By using genetic, dominant-negative and pharmacologic inhibitors, it can be shown that Cyclin-dependent Kinase 9 (CDK9) is necessary for hypertrophy in cardiomyocytes in vitro.

Heart-specific activation of CDK9 is often sufficient to induce cardiac myocyte hypertrophy in mice (see M. Sano et al., “Activation and function of cyclin T-Cdk9—positive transcription elongation factor-b—in cardiac muscle-cell hypertrophy”, Nat. Med. 2002, 11, 1310-1317; M. Sano, “Cyclins that don't cycle—cyclin T/cyclin-dependent kinase-9 determines cardiac muscle cell size”, Cell Cycle. 2003, 2, 99-104). In J. Vet. Med. Sci 2009, 71, 737-743, the effect of the flavonoid Quercetin on cardiac hypertrophy was shown.

Cardiac CDK9 appears to be initially activated by hypertrophic stimuli and seems to cause cardiac hypertrophy and heart failure. CDK9 is considered a relevant drug target for cardiac hypertrophy, either directly at the level of the kinase function or in the pathway leading to CDK9 activation. However, many drug compounds inhibit CDK9 and several other kinases as well, which can cause inconvenient side-effects.

In recent years, some traditional Chinese herbal remedies gained attention as new sources of drug compounds. Although their curative mechanisms are still largely unknown, some of the drugs have been used to treat cancer and other diseases. Extracts of the radix of the traditional Chinese herb “Huang-Qin” (Scutellaria baicalensis Georgi) are herbal remedies used in China for clinical treatment of diseases such as hyperlipidemia, atherosclerosis, hypertension, dysentery, common cold and atopic dermatitis. Huang-Qin extracts show a low toxicity in different animals. The active components of Huang-Qin extracts are specific flavonoids, in particular Wogonin.

The document WO 2000/44362 (D1) describes the use of flavopiridol derivatives for inhibiting smooth muscle cell (SMC) proliferation, e.g. after stent implantation.

The document EP-A 0366 061 describes 4-H-1-benzopyran-4-one derivatives and their use as pharmaceutically active agent, e.g. for inhibiting tumour growth. EP-A 0366 061 also describes the inhibition of oncogenic coded kinase by said compounds.

M. Yimam describes in “90-Day oral toxicity study of UP446, a combination of defined extracts of Scutellaria baicalensis and Acacia catechu, in rats” (FOOD AND CHEMICAL TOXICOLOGY, PERGAMON, GB, vol. 48, 2010, 5, pages 1202-1209) a toxicity study of standardized plant composition VP446. Said plant composition comprises several flavonoids.

The document WO 2006/099217 describes a pharmaceutical composition comprising a free-B-ring flavonoid, a flavan and at least one further agent, e.g. an anticoagulant.

The compound Wogonin (5,7-Dihydroxy-8-methoxy-flavone) is a specific flavone-derivative having the following chemical formula (W).

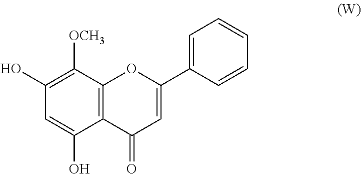 [SMILES: COc1c(O)cc(O)c2c(=O)cc(-c3ccccc3)oc12]

Wogonin (W) can be prepared by extraction or by chemical synthesis, e.g. by cyclization of 1,3-diaryl-diketons or by Wessely-Moser rearrangement. Wogonin is one of the major bioactive flavonoids of the plant Scutellaria Baicalensis Georgi, which has been shown to have anti-oxidant, anti-viral, anti-thrombotic and anti-inflammatory activities. Wogonin also shows cytostatic and pro-apoptotic effects on several tumour cells. Wogonin and structurally related natural flavones are inhibitors of cyclin-dependent kinase 9 (CDK9). Inhibition of CDK9 activity leads to reduced RNA synthesis and subsequently rapid down-regulation of the short-lived anti-apoptotic protein Mcl-1 resulting in apoptosis induction in cancer cells.

In mammals, e.g. humans, cardiac hypertrophy may occur as the result of a variety of reasons, including high blood pressure, valvular heart disease, myocardial infarction, and cardio-myopathy, and leads to an enlarged heart.

The presence of cardiac hypertrophy (on echocardiography (ECG)) is clinically important as it is often associated with increases in the incidence of heart failure, ventricular arrhythmias, death following myocardial infarction, decreased LV (left ventricular) ejection fraction, sudden cardiac death, aortic root dilation and a cerebro-vascular event. Cardiac hypertrophy also carries an increased risk for cardiac events such as angina, myocardial infarction, heart failure, serious ventricular arrhythmias and cardiovascular death. One of the typical signs of cardiac hypertrophy is an increase in the mass of the left ventricle (LV). This can be secondary to an increase in wall thickness and/or an increase in cavity size. Cardiac hypertrophy as a consequence of hypertension usually occurs with an increase in wall thickness, with or without an increase in cavity size.

The normal LV mass in men is 135 g and the mass index often is about 71 g/m². In women, the values are 99 g and 62 g/m², respectively. Left ventricle hypertrophy is usually defined as two standard deviations above normal. The typical echo-cardiographic criteria for left ventricle hypertrophy are ≧134 and 110 g/m² in men and women respectively (see Albergel Am. J. Cardiol. 1995, 75:498). In the clinical practice, the presence of left ventricle hypertrophy is more commonly defined by wall thickness values (obtained e.g. from M-mode or 2D images from the parasternal views). Hypertension associated cardiac hypertrophy may also result in interstitial fibrosis. Both factors contribute to an increase in left ventricular stiffness, resulting in diastolic dysfunction and an elevation in left ventricular end diastolic pressure.

Antihypertensive agents alone are not an effective treatment. Calcium channel blockers may e.g. increase risk in patients with abdominal aortic aneurysm. From a mechanistical point of view, many of the peptides and neurotransmitters that are implicated in the initiation and exacerbation of myocardial hypertrophy, including angiotensin II and endothelin, bind to cell membrane receptors which couple to a subset of intracellular heterotrimeric G proteins.

Many forms of heart diseases display cardiac hypertrophy, in particular an increase in the size of terminally differentiated cardio-myocytes. Cardiac hypertrophy occurs as a result of intrinsic haemodynamic stress, e.g. as a result of diminished heart function in myocardial infarction, or in response to extrinsic biomechanical stress. Normal cardiac growth after birth also involves hypertrophy. Gene expression profiles of neonatal and pathologically hypertrophic cardio-myocytes are similar. Both cell types are characterised by up-regulation of genes with functions in transcription, translation and cell survival.

Although the hypertrophic cardiac response is initially a beneficial adaptation to pathological stress from cardiovascular disease, in the longer term this response becomes de-compensated and can lead to heart failure at least in part through apoptotic and necrotic cell death. Hypertrophy increases the risk of cardiac morbidity and mortality.

Hypertrophy can be characterised by global increases in mRNA and protein synthesis. Main general drivers for transcription, TFIIH and P-TEFb, are closely associated with cardiac hypertrophy. Using genetic, dominant-negative and pharmacologic inhibitors, de-repression and activity of the enzyme cyclin-dependent Kinase 9 (CDK9) can be limiting for cardiac hypertrophy. The CDK9 activity seems necessary for hypertrophy in cardio-myocytes in vitro, and heart-specific activation of CDK9 by cyclin T1 seems to provoke hypertrophy in mice.

Some flavonoids, such as Quercetin and Chrysin, have recently been described to have some activity in hypertensive rats, but CDK9 inhibition has not been implicated in their beneficial effects.

It now was found that specific Wogonin derivatives, in particular compounds of the general formula (I), which inhibit the enzyme cyclin-dependent Kinase 9 (CDK9), can advantageously be used for the treatment of cardiac hypertrophy.

Of particular interest are compounds of general formula (I) and their pharmaceutically acceptable salts:

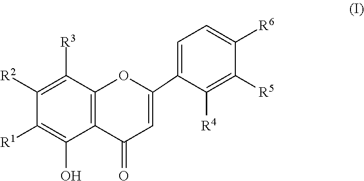

wherein:
R¹ is hydrogen, —CH₃, —CH₂OH, phenyl, hydroxyl-substituted phenyl,
R² is hydrogen, —CH₃, —OH, —CH₂OH, phenyl, hydroxyl-substituted phenyl,
R⁴ is hydrogen, —OH, —NH₂
R⁵ is hydrogen, —OH, in particular hydrogen
R⁶ is hydrogen, —OH, in particular hydrogen
R³ is —OCH₃ or a heterocyclic group (P)

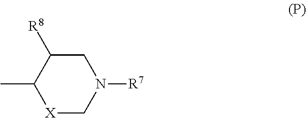

in which
X is —CH₂—, —O—, —CH(OH)—
R⁷ is hydrogen, —CH₃, —OCH₃
R⁸ is hydrogen, —OH.

The compound of formula (I) and their pharmaceutically acceptable salts, isomeric forms and polymorphic forms can be used for the prevention and therapy of cardiac hypertrophy. In particular, compounds of formula (I) seem to regulate the inflammation and to remodel what occurs in cardiac hypertrophy. The present invention is directed to compounds of formula (I) (and to pharmaceutical compositions containing such a compound) for preventing and/or treating cardiac hypertrophy.

One particular compound of formula (I) has already been described in the prior art, the compound Wogonin (W). Other compounds, in particular those, in which R³ denotes a heterocyclic group (P), have not yet been described in the literature, see e.g. the compounds described as Examples 2 to 17.

The invention relates to pharmaceutical compositions for use as a medicament for the treatment and/or prophylaxis of cardiac hypertrophy, comprising at least one compound of formula (I),

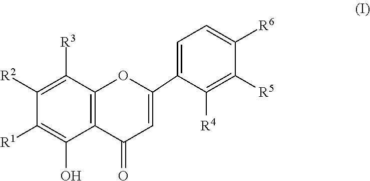

wherein:
R¹ is hydrogen, —CH₃, —CH₂OH, phenyl, hydroxyl-substituted phenyl,
R² is hydrogen, —CH₃, —OH, —CH₂OH, phenyl, hydroxyl-substituted phenyl,
R⁴ is hydrogen, —OH, —NH₂
R⁵ is hydrogen, —OH
R⁶ is hydrogen, —OH
R³ is —OCH₃ or a heterocyclic group (P)

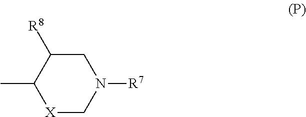

in which
R⁷ is hydrogen, —CH₃, —OCH₃
R⁸ is hydrogen, —OH
X is —CH₂, —CH(OH)—, —O—,
   or a pharmaceutically acceptable salt, an isomeric or polymorphic form thereof, and at least one pharmaceutically acceptable excipient. These excipients for various types of pharmaceutical compositions are known in the literature.

The pharmaceutical composition normally comprises from 0.1 to 2000 mg, often from 1 to 500 mg, of a compound of formula (I) and can e.g. be formulated as solid, liquid or semi-liquid formulation.

The invention also relates to pharmaceutical composition for use as a medicament for the treatment and/or prophylaxis of cardiac hypertrophy, wherein the composition comprises at least one compound of formula (I), wherein:
R¹ is hydrogen, —CH₃, —CH₂OH, phenyl
R² is hydrogen, —CH₃, —OH, —CH₂OH, phenyl
R⁴ is hydrogen, —OH, —NH₂
R⁵ is hydrogen,
R⁶ is hydrogen,
R³ is —OCH₃ or a heterocyclic group (P)

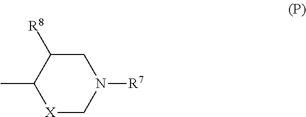

in which
R⁷ is hydrogen, —CH₃, —OCH₃
R⁸ is hydrogen, —OH
X is —CH₂, —CH(OH)—, —O—.
   or a pharmaceutically acceptable salt or an isomeric or polymorphic form thereof.

The invention also relates to a pharmaceutical composition for use as a medicament for the treatment and/or prophylaxis of cardiac hypertrophy, comprising at least one compound of formula (I), wherein the composition comprises at least one compound of formula (I), in which R³ denotes —OCH₃, or a pharmaceutically acceptable salt or an isomeric or polymorphic form thereof.

The invention also relates to a pharmaceutical composition for use as a medicament for the treatment and/or prophylaxis of cardiac hypertrophy, comprising from 0.1 to 500 mg of the compound Wogonin (W) and at least one pharmaceutically acceptable excipient.

A further aspect of the invention relates to a pharmaceutical composition comprising at least one compound of formula (I),

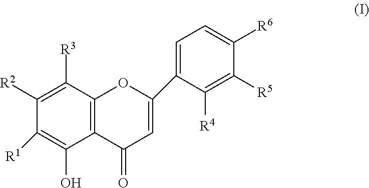

wherein:
R¹ is hydrogen, —CH₃, —CH₂OH, phenyl, hydroxyl-substituted phenyl,
R² is hydrogen, —CH₃, —OH, —CH₂OH, phenyl, hydroxyl-substituted phenyl,
R⁴ is hydrogen, —OH, —NH₂
R⁵ is hydrogen, —OH
R⁶ is hydrogen, —OH
R³ is —OCH₃ or a heterocyclic group (P)

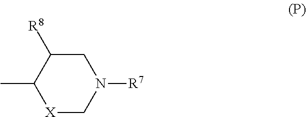

in which
R⁷ is hydrogen, —CH₃, —OCH₃
R⁸ is hydrogen, —OH
X is —CH₂, —CH(OH)—, —O—,
   or a pharmaceutically acceptable salt, an isomeric or polymorphic form thereof, and comprising a second drug compound and at least one pharmaceutically acceptable excipient.

This pharmaceutical composition can comprise as compound of formula (I) the drug compound Wogonin (W) and as second drug compound at least one from the following group: ACE inhibitors, beta blockers, anti-hypertensives, cardiotonics, anti-thrombotics, vasodilators, hormone antagonists, ionotropes, diuretics, endothelin antagonists, calcium channel blockers, phosphodiesterase inhibitors, angiotensin type II antagonists and cytokine inhibitors.

The pharmaceutical composition can e.g. comprise as compound of formula (I) the drug compound Wogonin (W) and as second drug compound an ACE inhibitor from the group ramipril, alacepril, enalapril, captopril, cilazapril, delapril, enalaprilat, fosinopril, lisinopril, moveltopril, perindopril and quinapril or the second drug compound ranolazin. The pharmaceutical composition can also comprise e.g. from 1 to 500 mg compound Wogonin (W) and as second drug compound an 1 to 50 mg of the ACE inhibitor ramipril or enalapril.

The invention also relates to new chemical compounds of formula (I)

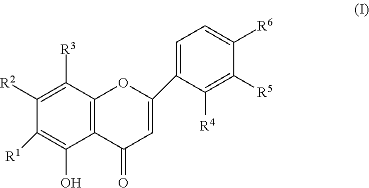

wherein:
R¹ is hydrogen, —CH₃, —CH₂OH, phenyl, hydroxyl-substituted phenyl,
R² is hydrogen, —CH₃, —OH, —CH₂OH, phenyl, hydroxyl-substituted phenyl,
R⁴ is hydrogen, —OH, —NH₂
R⁵ is hydrogen, —OH
R⁶ is hydrogen, —OH
R³ is a heterocyclic group (P)

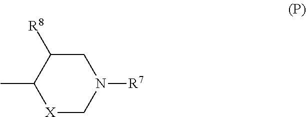

in which R⁷ is hydrogen, —CH₃, —OCH₃
   R⁸ is hydrogen, —OH
   X is —CH₂, —CH(OH)—, —O—,
      and pharmaceutically acceptable salts, isomeric forms and polymorphic forms thereof. The invention also relates to those compounds, in which R⁷ is hydrogen, —CH₃ or —OCH₃, R⁸ is hydrogen or —OH and X is —CH(OH)— or —O—.

The term “excipient” means a pharmaceutically inactive substance which is used as a carrier for the active substance and the design of formulations of drug products. The term “excipient” also encompasses a pharmaceutically acceptable, pharmacologically inactive ingredient such as a binder, a filler, a coating-forming compound, a plasticizers for coatings and a compound which masks odors. Examples of optional excipients are pigments, flavors, sweeteners, opacifiers, anti-adhesives, preservatives, glidants, lubricants and sorbents. Suitable substances are known in the art. The term “excipient” applied to pharmaceutical formulations of the invention also refers to a diluent or vehicle with which an active substance is administered. Such pharmaceutical excipient can be from animal, vegetable or synthetic origin, see also A. R. Gennaro, 20^{th} Edition in “Remington: The Science and Practice of Pharmacy”.

The term “pharmaceutically acceptable” in connection with a substance encompasses an ingredient or a substance which does not affect the safety of a human being and/or is well-tolerated by a human being after administration. The term “polymorphic form” encompasses an active substance, a pharmaceutically acceptable salt, solvate or isomer thereof forming different crystal structures or lattices.

A further aspect is a process for the preparation of a pharmaceutical composition, which comprises the step of mixing a compound of formula (I) or a pharmaceutically acceptable salt, an isomeric or polymorphic form thereof, at least one pharmaceutically acceptable excipient and (optionally) a second drug compound.

The invention also relates to a process for the preparation of a pharmaceutical composition, which comprises the step of mixing Wogonin (W), at least one pharmaceutically acceptable excipient and a second drug compound from the group: ACE inhibitors, beta blockers, anti-hypertensives, cardiotonics, anti-thrombotics, vasodilators, hormone antagonists, ionotropes, diuretics, endothelin antagonists, calcium channel blockers, phosphodiesterase inhibitors, angiotensin type II antagonists and cytokine inhibitors.

The treatment of cardiac hypertrophy can comprise the steps of first identifying a subject suffering from cardiac hypertrophy, then administering to the subject an effective amount of a compound of formula (I) to treat cardiac hypertrophy.

The amount of compound (I) to be applied depends on the individual to be treated, the dosage form, the specific medical status and other factors. A typical daily dose for prevention or treatment of cardiac hypertrophy with Wogonin or a compound of formula (I) would be in the range of 0.01 to 60 mg/kg. Wogonin (W) and the compounds of formula (I) are particularly useful for long term treatment of humans.

Positive signs of efficacy for treating cardiac hypertrophy by the present invention include demonstrable improvement in measurable signs, symptoms and other variables in test animals and in clinically relevant variables for cardiac hypertrophy. Typical signs of improvement include rapid regression or complete reversal of cardiac hypertrophy over a period of a few months, gradual regression of cardiac hypertrophy, the relative reduction in left ventricular mass index, cardiac function after regression of cardiac hypertrophy, reduced number of ventricular premature beats, reduced vulnerability to inducible ventricular fibrillation, reduced evidence of diastolic dysfunction, reduced risk of cardiovascular morbidity, reduced risk of cardiovascular mortality, and increase in general quality of life. Parameters for measurement of efficacy of the drug treatment include the reduction in left ventricular mass measured by echocardiography, reduction in wall thickness values obtained from 2D images from the parasternal views and number of ventricular premature beats.

The invention also relates to a combination of compound Wogonin (W) or of a derivative of formula (I) with one or several other drug compounds. Thus, in addition to the use described above, one may also provide to the patient other pharmaceutical cardiac therapies. The two drug compounds can be administered together or separately. They can be formulated together or be provided in different formulations.

Typical examples of drug compounds (“second drug compound”) to be used in combination with a compound of formula (I) include ACE inhibitors, beta blockers, anti-hypertensives, cardiotonics, anti-thrombotics, vasodilators, hormone antagonists, ionotropes, diuretics, endothelin antagonists, calcium channel blockers, phosphodiesterase inhibitors, angiotensin type II antagonists and cytokine inhibitors.

Particularly the combination with an ACE inhibitor (such as ramipril) or with a compound reducing the Na-ion flow into the heart muscle (such as ranolazin) are of interest.

Combinations of the pharmaceutically active compounds can be achieved by contacting cardiac cells (the individual) with a single pharmacological composition that includes both, compound (I) and the second drug compound, or by contacting the cell with two distinct compositions at the same time, wherein one composition includes a compound of formula (I) and the other includes the second drug compound. Alternatively, the application of a compound of formula (I) may precede or follow administration of the second drug compound by intervals ranging from minutes to weeks. When the compound of formula (I) and the second compound are applied separately, one generally ensures that a significant period of time did not expire between the times of each delivery, such that the compound (I) and the other drug would still be able to exert an advantageously combined effect on the cells. Typically administration with both compounds would be within 12 hours of each other, preferably within about 0.2 to 5 hours of each other. In some situations, it may be desirable to extend the time period for treatment significantly. It also is possible that more than one administration of either compound (I) or the other drug compound will be desired. In this regard, various combinations may be used. Where the compound (I) is “A” and the second drug compound is “B,” the following permutations based on 3 and 4 total administrations are exemplary:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B.

The typical daily dosage for treatment with Wogonin or a compound of formula (I) in combination with a second drug compound can be in the range of 0.01 to 60 mg/kg of compound (I) and 0.003 to 100 mg/kg of the second drug compound.

Typical examples of the second drug compound to be combined with a compound of formula (I), such as compound (W), are mentioned in the following.

a) The compound of formula (I) can be combined with an anti-hyperlipoproteinemics Administration of an agent that lowers the concentration of blood lipids and/or lipoproteins (“antihyperlipoproteinemic”) may be combined with a compound (1). An antihyperlipoproteinemic agent may e.g. be an aryloxyalkanoic/fibric acid derivative, a resin/bile acid sequesterant, a HMG CoA reductase inhibitor, a nicotinic acid derivative, a thyroid hormone or thyroid hormone analogy, a miscellaneous agent or a combination thereof.
Examples of aryloxyalkanoic/fibric acid derivatives include beclobrate, enzafibrate, binifibrate, ciprofibrate, clinofibrate, clofibrate (atromide-S), do fibric acid, etofibrate, fenofibrate, gemfibrozil (lobid), nicofibrate, pirifibrate, ronifibrate, simfibrate and theofibrate.
Examples of resins/bile acid sequesterants include cholestyramine (cholybar, questran), colestipol (colestid) and polidexide.
Examples of HMG CoA reductase inhibitors include lovastatin (mevacor), pravastatin (pravochol) or simvastatin (zocor).
Examples of nicotinic acid derivatives include nicotinate, acepimox, niceritrol, nicoclonate, nicomol and oxiniacic acid.
Examples of thyroid hormones and analogy thereof include etoroxate, thyropropic acid and thyroxine.
Examples of miscellaneous antihyperlipoproteinemics include acifran, azacosterol, benfluorex, β-benzalbutyramide, carnitine, chondroitin sulfate, clomestrone, detaxtran, dextran sulfate sodium, 5,8,11,14,17-eicosapentaenoic acid, eritadenine, furazabol, meglutol, melinamide, mytatrienediol, ornithine, y-oryzanol, 10 pantethine, pentaerythritol tetraacetate, a-phenylbutyramide, pirozadil, probucol (lorelco), β-sitosterol, sultosilic acid-piperazine salt, tiadenol, triparanol and xenbucin.

b) The compound of formula (I) can be combined with an anti-arteriosclerotic drug, such as a pyridinol carbamate.

c) In one embodiment, the compound of formula (I) is combined with antithrombotic and/or fibrinolytic agents, including anticoagulants, anticoagulant antagonists, antiplatelet agents, thrombolytic agents, thrombolytic agent antagonists or combinations thereof.
Examples of an anticoagulants include acenocoumarol, ancrod, anisindione, bromindione, clorindione, coumetarol, cyclocumarol, dextran sulphate, sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin.
Examples of antiplatelet agents include aspirin, a dextran, dipyridamole (persantin), heparin, sulfinpyranone (anturane) and ticlopidine (ticlid).
Examples of thrombolytic agents include tissue plaminogen activator (activase), plasmin, pro-urokinase, urokinase (abbokinase) streptokinase (streptase), anistreplase/APSAC (eminase).

d) In one embodiment, the compound of formula (I) is combined with a compound that may enhance blood coagulation. Examples of a blood coagulation promoting agent include thrombolytic agent antagonists and anticoagulant antagonists.
Examples of anticoagulant antagonists include protamine and vitamin Kl.
Examples of thrombolytic agent antagonists include amiocaproic acid (amicar) and tranexamic acid (amstat). Non-limiting examples of antithrombotics include anagrelide, argatroban, cilstazol, daltroban, defibrotide, enoxaparin, fraxiparine, indobufen, lamoparan, ozagrel, picotamide, plafibride, tedelparin, ticlopidine and triflusal.

e) In one embodiment, the compound of formula (I) is combined with antiarrhythmic compounds, including Class I antiarrythmic agents (sodium channel blockers), Class II antiarrythmic agents (β-adrenergic blockers), Class II antiarrythmic agents (repolarization prolonging drugs), Class IV antiarrhythmic agents (calcium channel blockers) and miscellaneous antiarrythmic agents.
Examples of sodium channel blockers include Class IA, Class IB and Class IC anti-arhythmic agents. Non-limiting examples of Class IA antiarrhythmic agents include disppyramide (norpace), procainamide (pronestyl) and 5 quinidine (quinidex). Non-limiting examples of Class IB antiarrhythmic agents include lidocaine (xylocalne), tocamide (tonocard) and mexiletine (mexitil). Non-limiting examples of Class IC antiarrhythmic agents include encamide (enkaid) and flecamide (tambocor).
Examples of a β blocker, otherwise known as a β-adrenergic blocker, a β-adrenergic antagonist or a Class II antiarrhythmic agent, include acebutolol (sectral), alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfinalol, talinolol, tertatolol, timolol, toliprolol and xibinolol. In certain aspects, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol.
Examples of an agent that prolong repolarization, also known as 30 a Class III antiarrhythmic agent, include amiodarone (cordarone) and sotalol (betapace).
Examples of a calcium channel blocker, otherwise known as a Class IV antiarrhythmic agent, include an arylalkylamine (e.g., bepridile, diltiazem, fendiline, gallopamil, prenylamine, terodiline, verapamil), a dihydropyridine 5 derivative (felodipine, isradipine, nicardipine, nifedipine, nimodipine, nisoldipine, nitrendipine) a piperazinde derivative (e.g., cinnarizine, flunarizine, lidoflazine) or a micellaneous calcium channel blocker such as bencyclane, etafenone, magnesium, mibefradil or perhexyline. In certain embodiments a calcium channel blocker comprises a long-acting dihydropyridine (nifedipine-type) calcium antagonist.
Examples of miscellaneous antiarrhymic agents include adenosine (adenocard), digoxin (lanoxin), acecamide, ajmaline, amoproxan, aprindine, bretylium tosylate, bunaftine, butobendine, capobenic acid, cifenline, 15 disopyranide, hydroquinidine, indecamide, ipatropium bromide, lidocaine, lorajmine, lorcamide, meobentine, moricizine, pirmenol, prajmaline, propafenone, pyrinoline, quinidine polygalacturonate, quinidine sulfate and viquidil.

f) In one embodiment, the compound of formula (I) is combined with antihypertensive agents, including sympatholytic, alpha/beta blockers, alpha blockers, anti-angiotensin II agents, beta blockers, calcium channel blockers, vasodilators and miscellaneous antihypertensives.
Examples of an α-blocker, also known as an α-adrenergic blocker or an α-adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin.
Sometimes, an antihypertensive agent is both an alpha and beta-adrenergic antagonist. Examples of an α/β blocker comprise labetalol (normodyne, trandate).
Examples of anti-angiotensin II agents include angiotensin converting enzyme inhibitors and angiotensin II receptor antagonists. Examples of angiotensin converting enzyme inhibitors (ACE inhibitors) include alacepril, enalapril, captopril, cilazapril, delapril, enalaprilat, fosinopril, lisinopril, moveltopril, perindopril, quinapril and ramipril. The combination of Wogonin and ramipril is of particular interest. Examples of an angiotensin II receptor blocker, also known as an angiotensin II receptor antagonist, an ANG receptor blocker or an ANG-II type-1 receptor blocker (ARBS), include angiocandesartan, eprosartan, irbesartan, losartan and valsartan.
Examples of a sympatholytic include a centrally acting sympatholytic or a peripherially acting sympatholytic. Examples of a centrally acting sympatholytic, also known as a central nervous system (CNS) sympatholytic, include clonidine (catapres), guanabenz (wytensin) guanfacine (tenex) and methyldopa (aldomet). Examples of a peripherally acting sympatholytic include a ganglion blocking agent, an adrenergic neuron blocking agent, a β-adrenergic blocking agent or an al-adrenergic blocking agent. Examples of a ganglion blocking agent include mecamylamine (inversine) and trimethaphan (arfonad). Non-limiting of an adrenergic neuron blocking agent include guanethidine (ismelin) and reserpine (serpasil). Examples of a β-adrenergic blocker include acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren). Examples of al-adrenergic blocker include prazosin (minipress), doxazocin (cardura) and terazosin (hytrin).
The cardiovasculator agent may comprise a vasodilator (e.g., a cerebral vasodilator, a coronary vasodilator or a peripheral vasodilator). In certain embodiments, a vasodilator comprises a coronary vasodilator. Examples of a coronary vasodilator include amotriphene, bendazol, benfurodil hemisuccinate, benziodarone, chloracizine, chromonar, clobenfurol, clonitrate, dilazep, dipyridamole, droprenilamine, efloxate, erythrityl tetranitrane, etafenone, fendiline, floredil, ganglefene, herestrol bis(β-diethylaminoethyl ether), hexobendine, itramin tosylate, khellin, lidoflanine, mannitol, hexanitrane, medibazine, nicorglycerin, pentaerythritol tetranitrate, pentrinitrol, perhexyline, pimethylline, trapidil, tricromyl, trimetazidine, troInitrate phosphate and visnadine.
A vasodilator may comprise a chronic therapy vasodilator or a hypertensive emergency vasodilator. Examples of a chronic therapy vasodilator include hydralazine (apresoline) and minoxidil (loniten). Examples of a hypertensive emergency vasodilator include nitroprusside (nipride), diazoxide (hyperstat IV), hydralazine (apresoline), minoxidil (loniten) and verapamil.
Examples of miscellaneous antihypertensives include ajmaline, γ-aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil.
The antihypertensive may comprise an arylethanolamine derivative, a benzothiadiazine derivative, a N-carboxyalkyl(peptide/lactam) derivative, a dihydropyridine derivative, a guanidine derivative, a hydrazines/phthalazine, an imidazole derivative, a quanternary ammonium compound, a reserpine derivative or a suflonamide derivative.
Examples of arylethanolamine derivatives include amosulalol, bufuralol, dilevalol, labetalol, pronethalol, sotalol and sulfinalol.
Examples of benzothiadiazine derivatives include althizide, bendrofiumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydrofiumethizide, methyclothiazide, meticrane, metolazone, paraflutizide, polythizide, tetrachlormethiazide and trichlormethiazide.
Examples of N-carboxyalkyl(peptide/lactam) derivatives include alacepril, captopril, cilazapril, delapril, enalapril, enalaprilat, fosinopril, lisinopril, moveltipril, perindopril, quinapril and ramipril.
Examples of dihydropyridine derivatives include amlodipine, felodipine, isradipine, nicardipine, nifedipine, nilvadipine, nisoldipine and nitrendipine.
Examples of guanidine derivatives include bethanidine, debrisoquin, guanabenz, guanacline, guanadrel, guanazodine, guanethidine, guanfacine, guanochlor, guanoxabenz and guanoxan.
Examples of hydrazines/phthalazines include budralazine, cadralazine, dihydralazine, endralazine, hydracarbazine, hydralazine, pheniprazine, pildralazine and todralazine.
Examples of imidazole derivatives include clonidine, lofexidine, phentolamine, tiamenidine and tolonidine.
Examples of quanternary ammonium compounds include azamethonium bromide, chlorisondamine chloride, hexamethonium, pentacynium bis(methylsulfate), penta-methonium bromide, pentolinium tartrate, phenactropinium chloride and trimethidinium methosulfate.
Examples of reserpine derivatives include bietaserpine, deserpidine, rescinnamine, reserpine and syrosingopine.
Examples of sulfonamide derivatives include ambuside, clopamide, furosemide, indapamide, quinethazone, tripamide and xipamide.
Examples of a vasopressor, also known as an antihypotensive, include amezinium methyl sulfate, angiotensin amide, dimetofrine, dopamine, etifelmin, etilefrin, gepefrine, metaraminol, midodrine, norepinephrine, pholedrine and synephrine.
Examples of agents for the treatment of congestive heart failure include anti-angiotension II agents, afterload-preload reduction treatment, diuretics and inotropic agents.

Examples of a diuretic include a thiazide or benzothiadiazine derivative althiazide, bendroflumethazide, benzthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furtherene, triamterene), purines (e.g., acefylline, 7-morpholino-methyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, do fenamide, clopamide, clorexolone, diphenylmethane-4,4′-disulfonamide, disulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene) or a miscellaneous diuretic such as aminozine, arbutin, chlorazanil, ethacrynic acid, etozolin, hydracarbazine, isosorbide, mannitol, metochalcone, muzolimine, perhexyline, ticrnafen and urea.
Examples of a positive inotropic agent, also known as a cardiotonic, include acefylline, an acetyldigitoxin, 2-amino-4-picoline, aminone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.
In particular aspects, an inotropic agent is a cardiac glycoside, a beta-adrenergic agonist or a phosphodiesterase inhibitor. Examples of a cardiac glycoside include digoxin (lanoxin) and digitoxin (crystodigin). Examples of a β-adrenergic agonist include albuterol, bambuterol, bitolterol, carbuterol, clenbuterol, clorprenaline, denopamine, dioxethedrine, dobutamine, (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Examples of a phosphodiesterase inhibitor include aminone (inocor).
Antianginal agents may comprise organonitrates, calcium channel blockers, beta blockers and combinations thereof. Examples of organonitrates, also known as nitrovasodilators, include nitroglycerin (nitro-bid, nitrostat), isosorbide dinitrate (isordil, sorbitrate) and amyl nitrate (aspirol, vaporole).

g) The compound of formula (I) can in particular be combined with ranolazin, which reduces the Na⁺-flow into the cardiac cell.

The compounds of formula (I) are well tolerated in animals (including humans) and can be used in various pharmaceutical compositions. Generally, for the preparation of compositions, pharmaceutically acceptable excipients are used. In general these excipients (auxiliaries) are essentially free of pyrogens and other impurities that could be harmful to humans or animals.

Often, appropriate salts and buffers are used in the compositions to render the drug compound (I) or drug compounds stable and to allow for uptake by target heart muscle cells. Aqueous compositions of the present invention comprise an effective amount of the compound(s), dissolved or dispersed in a pharmaceutically acceptable carrier (excipient) or aqueous medium. The term “pharmaceutically or pharmacologically acceptable” refer to molecular entities and compositions that do not produce adverse, allergic, or other unwanted reactions when administered to an animal or a human.

As used herein, “pharmaceutically acceptable excipient” includes solvents, buffers, solutions, dispersion media, coatings, antibacterial and antifungal agents, isotonic and absorption delaying agents etc. acceptable for use in formulating pharmaceuticals.

Second active ingredients also can be incorporated into the compositions, provided they do not inactivate the compound of formula (I) of the compositions.

The pharmaceutical compositions of the present invention include all types of classical pharmaceutical preparations. Administration of these compositions according to the present invention may be via any common route as long as the cardiac tissue is available via that route. This includes oral, parenteral, sublingual, nasal and buccal application. Administration may also be by intradermal, subcutaneous, intramuscular, intraperitoneal or intravenous injection.

The compounds of formula (I), in particular Wogonin, may also be administered parenterally or intraperitoneally. By way of illustration, solutions of the active compounds as free base or pharmacologically acceptable salts can be prepared in water suitably mixed with an excipient, e.g. a surfactant, such as hydroxypropylcellulose. Dispersions can also be prepared in glycerol, liquid polyethylene glycols, and mixtures thereof and in oils. For ordinary conditions of storage and use, these compositions can contain a preservative to prevent the growth of microorganisms.

The pharmaceutical forms suitable for injectable use of formula (I) compounds include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents. Examples are: parabens, chlorobutanol, phenol, sorbic acid and thimerosal. It can be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the compounds of formula (I) in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

For oral administration the compound of formula (I) generally may be incorporated with excipients and used in the form of non-ingestible mouthwashes and dentifrices.

The compositions of the present invention generally may be formulated in a neutral or salt form. Pharmaceutically-acceptable salts include, for example, acid addition salts derived from inorganic acids (e.g., hydrochloric or phosphoric acids), or from organic acids (e.g., acetic, oxalic, tartaric, mandelic acid). Salts formed can also be derived from inorganic bases (e.g., sodium, potassium, ammonium, calcium, or ferric hydroxides) or from organic bases (e.g., isopropylamine, trimethylamine, histidine and procaine).

Upon formulation, solutions are preferably administered in a manner compatible with the dosage formulation and in such amount as is therapeutically effective. The formulations may easily be administered in a variety of dosage forms such as injectable solutions, drug release capsules and tablets. For parenteral administration in an aqueous solution, for example, the solution generally is suitably buffered and the liquid diluent first rendered isotonic for example with sufficient saline or glucose. Such aqueous solutions may e.g. be used for intravenous, intramuscular, subcutaneous and intraperitoneal administration. Preferably, sterile aqueous media are employed as is known to those of skill in the art. By way of illustration, a single dose of compound (1) may be dissolved in 1 ml of isotonic NaCl solution and injected at the proposed site of infusion, (see for example, “Remington's Pharmaceutical Sciences” 15th Edition). Some variation in dosage will necessarily occur depending on the condition of the subject being treated.

The person responsible for administration will determine the appropriate dose for the individual subject. Moreover, for human administration, preparations should meet sterility, pyrogenicity, safety and purity standards as required by national or international standards.

The invention is further illustrated by the following examples.

### EXAMPLE 1a

### Preparation of Wogonin (W)

500 mg of the compound Wogonin were prepared by chemical synthesis.

The compound (W) was purified by classical crystallisation and/or chromatography.

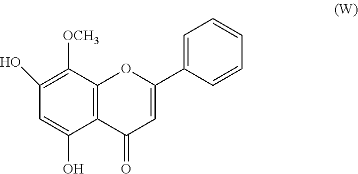 [SMILES: COc1c(O)cc(O)c2c(=O)cc(-c3ccccc3)oc12]

### EXAMPLE 1b

### Wogonin Inhibition of the Enzyme CDK9

To determine the effect of Wogonin on the enzymatic activity of CDK9, CDK9/cyclinT (Millipore, Dundee, UK) and the substrate peptide (Millipore) were incubated with 8 mM MOPS pH 7.0, 0.2 mM EDTA, 10 mM Mg-acetate and 10 μM γ-³³-P-ATP. The reaction was initiated by the addition of the MgATP mix. After incubation for 40 min at room temperature, the reaction was stopped by the addition of a 3% phosphoric acid solution. An aliqot of the reaction was then spotted onto a P30 filtermat and washed three times for 5 min in 75 mM phosphoric acid and once in methanol prior to drying and scintillation counting. Alternatively, the inhibitory effect of Wogonin on kinase activity was examined by incubating recombinant CDK9/cyclinT (Proqinase GmbH, Freiburg, Germany) and recombinant substrate RBER-CHKtide (Proqinase) in the presence of different concentrations of Wogonin in 60 mM HEPES-NaOH, pH 7.5, 3 mM MgCl₂, 3 mM MnCl₂, 3 μM Na-orthovanadate, 1.2 mM DTT, 50 μg/ml PEG₂₀₀₀₀ and 1 μM ATP for 2 h at 30° C.

The amount of phosphorylated substrate was determined by Western Blot using phospho-specific antibodies. Wogonin was found to be a very potent inhibitor (IC₅₀<0.2 μM) for CDK9. In contrast to other known flavone-derivatives, Wogonin (and the compounds of formula I) strongly inhibits the enzyme CDK9, but does not strongly inhibit many other important cellular kinases, such as CDK2, CDK4 and CDK6.

It also can be observed that Wogonin (and other compounds of formula I) do not inhibit the kinases CDK4 and CDK6 (applied at a dose of 50 μM). Contrary to this, the known compound Quercetin does inhibit the kinases CDK4 and CDK6 (applied at a dose of 50 M).

Furthermore it can be found that Wogonin applied for 48 hours to T-cells leads to much less DNA-fragmentation (applied at doses of 10 μM, 50 μM and 100 μM) than the known compound Quercetin. At a dose of 10 μM, Wogonin showed less than 5% of DNA-fragmentation, whereas Quercetin showed more than 15% of DNA-fragmentation. This shows the low toxicity of Wogonin which is a great advantageous for a long-term application as drug compound.

Furthermore it can be found that Wogonin has a much longer inhibitory effect on the enzyme CDK9 (applied at a dose of 50 μM in CEM) than the known compound Quercetin.

### EXAMPLE 1c

### Application of Wogonin in Ventricular Cardiac Myocytes

Wogonin (W) as a major bioactive constituent of the traditional Chinese herb Scutellaria can be used for prevention and treatment of cardiac hypertrophy. To test Wogonin in hypertrophic conditions, ventricular cardiac myocytes from neonatal rats were isolated.

Hypertrophy was induced by stimulating the cells (cardiomyocytes) with phenylephrine. The experiments performed in primary rat cells showed that phenylephrine-triggering resulted in a significant elevation of phosphorylation of the RNAPII C-terminal domain at the position Ser². Treatment of the cells with Wogonin (50 μM in a solution) almost completely inhibited the phenylephrine-induced phosphorylation of RNAPII.

Furthermore, Wogonin is well supported by the cells, even at higher doses. A comparative compound, Flavopiridol, a semi-synthetic pan-inhibitor of CDKs, was shown to also inhibit CDK9. Flavopiridol inhibits phosphorylation of the RNAPII C-terminal domain at the position Ser². However, Flavopiridol is more toxic to normal lymphoid cells when used at higher concentrations. The toxicities of Wogonin as compared to Flavopiridol were tested in normal and malignant T lymphocytes. Up to concentrations of 100 μM, Wogonin was found to have no or very low toxicity to normal proliferating T cells in comparison to leukemic CEM cells. Contrary to this, Flavopiridol at concentrations of e.g. 500 nM required to kill leukemic cells was found to be toxic to normal cells.

T-lymphocytes isolated from 3-6 healthy donors were activated through PHA stimulation and further cultured for 5 days (called d6 T cells) in medium supplemented with IL-2. d6 T-cells and leukemic CEM T-cells were treated with different concentrations of Wogonin (the resulting concentration in the solution was from 0 μM to 100 μM) or Flavopiridol (the resulting concentration in the solution was from 0 nM to 1000 nM). Apoptotic cell death was determined by DNA fragmentation after 48 hours.

Several other flavones of general formula (I) can also strongly inhibit CDK9 activity. The toxicities of these flavones to normal proliferating lymphocytes were tested. T lymphocytes were isolated from healthy donors.

Since proliferating cells are in general more susceptible to drug-induced apoptotic cell death, the T cells were activated through PHA stimulation and cultured for further 5 days under supplementation of IL-2 (so called d6 T cells). Treatment of normal proliferating T cells with different compounds of general formula (I) showed that Wogonin has a particularly low toxicity to normal T cells compared to other flavones tested.

Furthermore, Wogonin can be used in combination with other drug compounds known for treatment of cardiovascular diseases. E.g. the combination with an ACE inhibitor, such as Enalapril or Ramipril, or with the angina pectoris-drug Ranolazin, provides beneficial effects on heart diseases, in particular primary and secondary cardiac hypertrophy.

Other combination therapies with Wogonin and second drugs involve other sympatholytic compounds, such as α-blockers and β-blockers, anti-angiotensin II agents, calcium channel blockers and vasodilators. To investigate effects of Wogonin as single compound or in combination (e.g. with an ACE-inhibitor) in vivo, mouse cardiac hypertrophy models can be used. The experiments are carried out with Wogonin (e.g. 0.1 mg/Kg) or in combination with e.g. Ramipril (e.g. 0.06 mg/Kg).

### EXAMPLE 2

### Preparation of Compound (2)

In analogy to the flavone compound Wogonin, the following derivatives of formula (I) can be prepared by classical synthesis. They can be purified by chromatography.

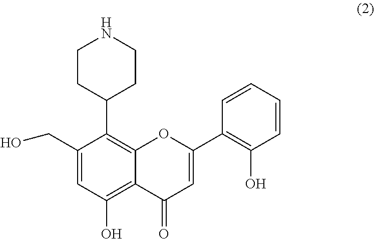 [SMILES: O=c1cc(-c2ccccc2O)oc2c(C3CCNCC3)c(CO)cc(O)c12]

### EXAMPLE 3

### Preparation of Compound (3)

In analogy to Wogonin, the following derivative of formula (I) can be prepared by classical synthesis

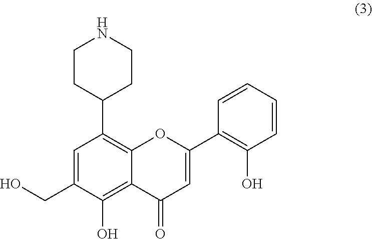 [SMILES: O=c1cc(-c2ccccc2O)oc2c(C3CCNCC3)cc(CO)c(O)c12]

### EXAMPLE 4

### Preparation of Compound (4)

In analogy to Wogonin, the following derivative of formula (I) can be prepared by classical synthesis

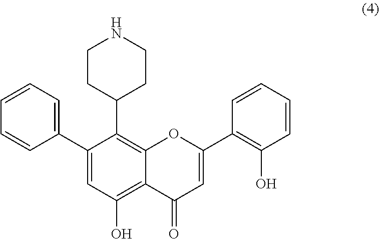 [SMILES: O=c1cc(-c2ccccc2O)oc2c(C3CCNCC3)c(-c3ccccc3)cc(O)c12]

### EXAMPLE 5

### Preparation of Compound (5)

In analogy to Wogonin, the following derivative of formula (I) can be prepared by classical synthesis

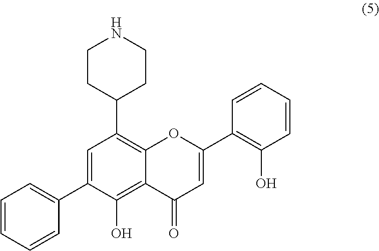 [SMILES: O=c1cc(-c2ccccc2O)oc2c(C3CCNCC3)cc(-c3ccccc3)c(O)c12]

### EXAMPLE 6

### Preparation of Compound (6)

In analogy to Wogonin, the following derivative of formula (I) can be prepared by classical synthesis

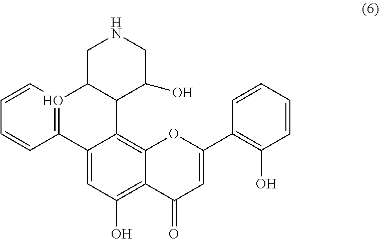

### EXAMPLE 7

### Preparation of Compound (7)

In analogy to Wogonin, the following derivative of formula (I) can be prepared by classical synthesis

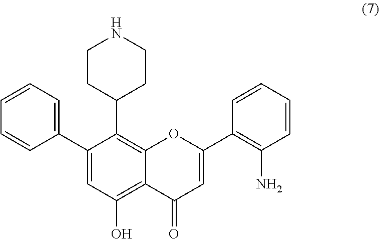 [SMILES: Nc1ccccc1-c1cc(=O)c2c(O)cc(-c3ccccc3)c(C3CCNCC3)c2o1]

### EXAMPLE 8

### Preparation of Compound (8)

In analogy to Wogonin, the following derivative of formula (I) can be prepared by classical synthesis

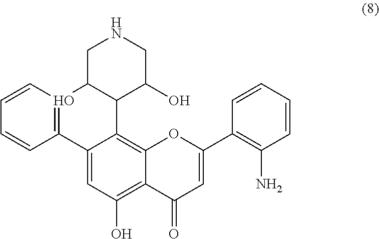

### EXAMPLE 9

### Preparation of Compound (9)

In analogy to Wogonin, the following derivative of formula (I) can be prepared by classical synthesis

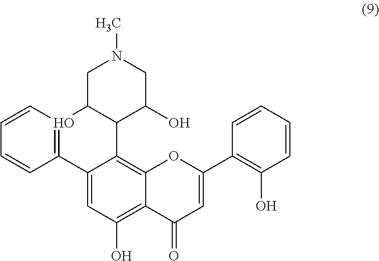

### EXAMPLE 10

### Preparation of Compound (10)

In analogy to Wogonin, the following derivative of formula (I) can be prepared by classical synthesis

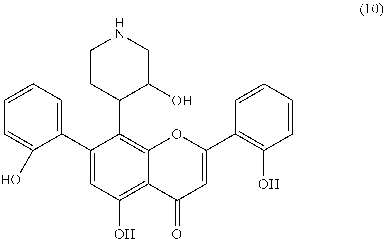 [SMILES: O=c1cc(-c2ccccc2O)oc2c(C3CCNCC3O)c(-c3ccccc3O)cc(O)c12]

### EXAMPLE 11

### Preparation of Compound (11)

In analogy to Wogonin, the following derivative of formula (I) can be prepared by classical synthesis

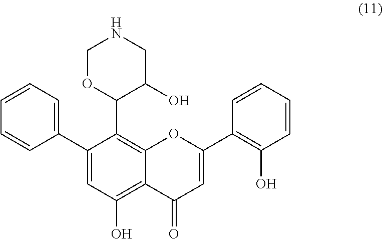 [SMILES: O=c1cc(-c2ccccc2O)oc2c(C3OCNCC3O)c(-c3ccccc3)cc(O)c12]

### EXAMPLE 12

### Preparation of Compound (12)

In analogy to Wogonin, the following derivative of formula (I) can be prepared by classical synthesis

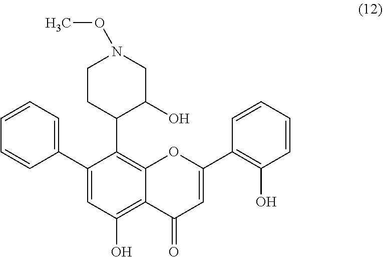 [SMILES: CON1CCC(c2c(-c3ccccc3)cc(O)c3c(=O)cc(-c4ccccc4O)oc23)C(O)C1]

### EXAMPLE 13

### Preparation of Compound (13)

In analogy to Wogonin, the following derivative of formula (I) can be prepared by classical synthesis

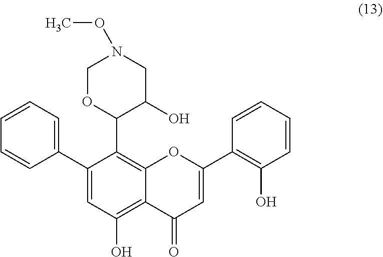 [SMILES: CON1COC(c2c(-c3ccccc3)cc(O)c3c(=O)cc(-c4ccccc4O)oc23)C(O)C1]

### EXAMPLE 14

### Preparation of Compound (14)

In analogy to Wogonin, the following derivative of formula (I) can be prepared by classical synthesis

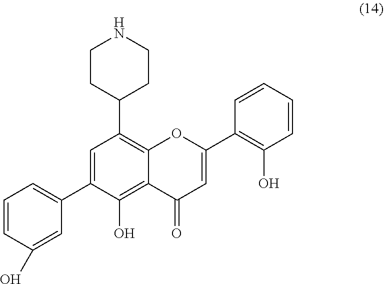 [SMILES: O=c1cc(-c2ccccc2O)oc2c(C3CCNCC3)cc(-c3cccc(O)c3)c(O)c12]

### EXAMPLE 15

### Preparation of Compound (15)

In analogy to Wogonin, the following derivative of formula (I) can be prepared by classical synthesis

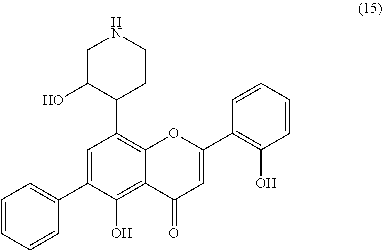 [SMILES: O=c1cc(-c2ccccc2O)oc2c(C3CCNCC3O)cc(-c3ccccc3)c(O)c12]

### EXAMPLE 16

### Preparation of Compound (16)

In analogy to Wogonin, the following derivative of formula (I) can be prepared by classical synthesis

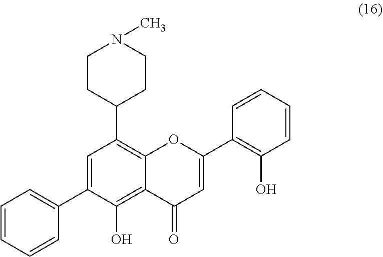 [SMILES: CN1CCC(c2cc(-c3ccccc3)c(O)c3c(=O)cc(-c4ccccc4O)oc23)CC1]

### EXAMPLE 17

### Preparation of Compound (17)

In analogy to Wogonin, the following derivative of formula (I) can be prepared by classical synthesis

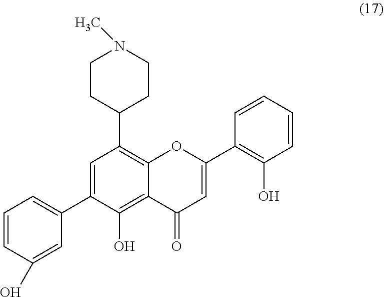 [SMILES: CN1CCC(c2cc(-c3cccc(O)c3)c(O)c3c(=O)cc(-c4ccccc4O)oc23)CC1]

These compounds (2) to (17) are promising inhibitors of CDK9 and can be used for the treatment of various diseases, such as cardiac hypertrophy.

1. A pharmaceutical composition for use as a medicament for the treatment and/or prophylaxis of cardiac hypertrophy, comprising at least one compound of formula (I), 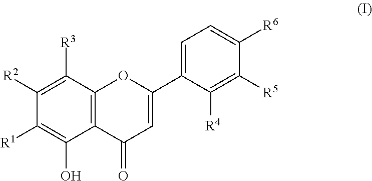
wherein:
R¹ is hydrogen, —CH₃, —CH₂OH, phenyl, or hydroxyl-substituted phenyl,
R² is hydrogen, —CH₃, —OH, —CH₂OH, phenyl, or hydroxyl-substituted phenyl,
R⁴ is hydrogen, —OH, or —NH₂
R⁵ is hydrogen, or —OH
R⁶ is hydrogen, or —OH
R³ is —OCH₃ or a heterocyclic group (P) 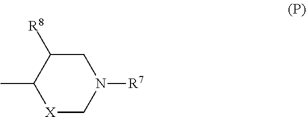
in which
R⁷ is hydrogen, —CH₃, or —OCH₃
R⁸ is hydrogen, or —OH
X is —CH₂, —CH(OH)—, or —O—,
or a pharmaceutically acceptable salt, an isomeric or polymorphic form thereof, and at least one pharmaceutically acceptable excipient.

2. A pharmaceutical composition for use as a medicament for the treatment and/or prophylaxis of cardiac hypertrophy according to claim 1, wherein the composition comprises at least one compound of formula (I), wherein:
R¹ is hydrogen, —CH₃, —CH₂OH, or phenyl
R² is hydrogen, —CH₃, —OH, —CH₂OH, or phenyl
R⁴ is hydrogen, —OH, or —NH₂
R⁵ is hydrogen,
R⁶ is hydrogen,
R³ is —OCH₃ or a heterocyclic group (P) 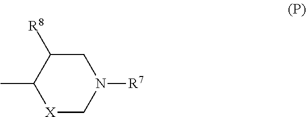
in which
R⁷ is hydrogen, —CH₃, or —OCH₃
R⁸ is hydrogen, or —OH
X is —CH₂, —CH(OH)—, or —O—.
or a pharmaceutically acceptable salt or an isomeric or polymorphic form thereof.

3. A pharmaceutical composition for use as a medicament for the treatment and/or prophylaxis of cardiac hypertrophy, comprising at least one compound of formula (I), according to claim 1, wherein the composition comprises at least one compound of formula (I), in which R³ denotes —OCH₃, or a pharmaceutically acceptable salt or an isomeric or polymorphic form thereof.

4. A pharmaceutical composition for use as a medicament for the treatment and/or prophylaxis of cardiac hypertrophy, according to claim 1, comprising from 0.1 to 2000 mg of the compound Wogonin (W) and at least one pharmaceutically acceptable excipient.

5. A pharmaceutical composition comprising at least one compound of formula (I), 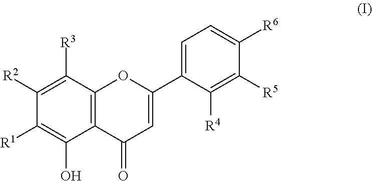
wherein:
R¹ is hydrogen, —CH₃, —CH₂OH, phenyl, or hydroxyl-substituted phenyl,
R² is hydrogen, —CH₃, —OH, —CH₂OH, phenyl, or hydroxyl-substituted phenyl,
R⁴ is hydrogen, —OH, or —NH₂
R⁵ is hydrogen, —OH
R⁶ is hydrogen, —OH
R³ is —OCH₃ or a heterocyclic group (P) 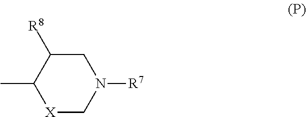
in which
R⁷ is hydrogen, —CH₃, or —OCH₃
R⁸ is hydrogen, or —OH
X is —CH₂, —CH(OH)—, or —O—,
or a pharmaceutically acceptable salt, an isomeric or polymorphic form thereof, and comprising a second drug compound and at least one pharmaceutically acceptable excipient.

6. A pharmaceutical composition according to claim 5, comprising as compound of formula (I) the drug compound Wogonin (W) and as second drug compound at least one from the following group: ACE inhibitors, beta blockers, anti-hypertensives, cardiotonics, anti-thrombotics, vasodilators, hormone antagonists, ionotropes, diuretics, endothelin antagonists, calcium channel blockers, phosphordiesterase inhibitors, angiotensin type II antagonists and cytokine inhibitors.

7. A pharmaceutical composition according to claim 5, comprising as compound of formula (I) the drug compound Wogonin (W) and as second drug compound an ACE inhibitor from the group ramipril, alacepril, enalapril, captopril, cilazapril, delapril, enalaprilat, fosinopril, lisinopril, moveltopril, perindopril and quinapril or the second drug compound ranolazin.

8. A pharmaceutical composition according to claim 5, comprising from 1 to 500 mg compound Wogonin (W) and as second drug compound an 1 to 50 mg of the ACE inhibitor ramipril or enalapril.

9. A compound of formula (I) 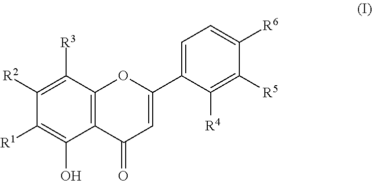
wherein:
R¹ is hydrogen, —CH₃, —CH₂OH, phenyl, or hydroxyl-substituted phenyl,
R² is hydrogen, —CH₃, —OH, —CH₂OH, phenyl, or hydroxyl-substituted phenyl,
R⁴ is hydrogen, —OH, or —NH₂
R⁵ is hydrogen, or —OH
R⁶ is hydrogen, or —OH
R³ is a heterocyclic group (P) 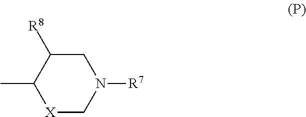
in which
R⁷ is hydrogen, —CH₃, or —OCH₃
R⁸ is hydrogen, or —OH
X is —CH₂, —CH(OH)—, or —O—,
or a pharmaceutically acceptable salt, an isomeric or polymorphic form thereof.

10. A process for the preparation of a pharmaceutical composition according to claim 5, which comprises the step of mixing a compound of formula (I) or a pharmaceutically acceptable salt, an isomeric or polymorphic form thereof, at least one pharmaceutically acceptable excipient and a second drug compound.

11. A process for the preparation of a pharmaceutical composition according to claim 10, which comprises the step of mixing Wogonin (W), at least one pharmaceutically acceptable excipient and a second drug compound from the group: ACE inhibitors, beta blockers, anti-hypertensives, cardiotonics, anti-thrombotics, vasodilators, hormone antagonists, ionotropes, diuretics, endothelin antagonists, calcium channel blockers, phosphordiesterase inhibitors, angiotensin type II antagonists and cytokine inhibitors.
